(19) United States
(12) Patent Application Publication
(10) Pub. No.: US 2009/0281430 A1
(43) Pub. Date: Nov. 12, 2009

(54) CATHETER WITH SPINNING ULTRASOUND TRANSCEIVER BOARD

(75) Inventor: Mark Wilder, Lexington, MA (US)

Correspondence Address: FISH & RICHARDSON PC, P.O. BOX 1022, MINNEAPOLIS, MN, 55440-1022, US

(73) Assignee: INFRAREDX, INC., Burlington, MA (US)

(21) Appl. No.: 12/437,114

(22) Filed: May 7, 2009

Related U.S. Application Data
(60) Provisional application No. 61/007,515, filed on May 7, 2008.

(51) Int. Cl. A61B 8/14

(52) U.S. Cl. 600/463

(57) ABSTRACT

An apparatus for detecting vulnerable plaque in a blood vessel includes an intravascular probe, and a slip ring at a proximal end of the probe. The slip ring has a stationary portion and a spinning portion. An ultrasound transceiver board is mechanically coupled to the slip ring's spinning portion for communication with an ultrasound transducer, also within the probe. A transmission line extends between the ultrasound transducer and the ultrasound transceiver board.

### RELATED APPLICATION

This application is a non-provisional claiming the benefit of the priority date of U.S. Application No. 61/007,515, filed May 7, 2008, the contents of which are incorporated herein by reference.

### TECHNICAL FIELD

The invention relates to vulnerable plaque detection, and in particular, to catheters used to detect vulnerable plaque.

### BACKGROUND

Atherosclerosis is a vascular disease characterized by a modification of the walls of blood-carrying vessels. Such modifications, when they occur at discrete locations or pockets of diseased vessels, are referred to as plaques. Certain types of plaques are associated with acute events such as stroke or myocardial infarction. These plaques are referred to as “vulnerable plaques.” A vulnerable plaque typically includes a lipid-containing pool separated from the blood by a thin fibrous cap. In response to elevated intraluminal pressure or vasospasm, the fibrous cap can become disrupted, exposing the contents of the plaque to the flowing blood. The resulting thrombus can lead to ischemia or to the shedding of emboli.

One method of locating vulnerable plaque is to peer through the arterial wall with infrared light. To do so, one inserts a catheter through the lumen of the artery. The catheter includes a delivery fiber for illuminating a spot on the arterial wall with infrared light. A portion of the light penetrates the blood and arterial wall, scatters off structures within the wall and re-enters the lumen. This re-entrant light can be collected by a collection fiber within the catheter and subjected to spectroscopic analysis. This type of diffuse reflectance spectroscopy can be used to determine chemical composition of arterial tissue, including key constituents believed to be associated with vulnerable plaque such as lipid content.

Another method of locating vulnerable plaque is to use intravascular ultrasound (IVUS) to detect the shape of the arterial tissue surrounding the lumen. To use this method, one also inserts a catheter through the lumen of the artery. The catheter includes an ultrasound transducer to send ultrasound energy towards the arterial wall. The reflected ultrasound energy is received by the ultrasound transducer and is used to map the shape of the arterial tissue. This map of the morphology of the arterial wall can be used to detect the fibrous cap associated with vulnerable plaque.

### SUMMARY

The invention arises in an effort to overcome noise and electromagnetic interference associated with transport of RF energy across a slip-ring that interfaces a spinning portion of a catheter with stationary elements that generate and/or process the RF energy.

In one aspect, the invention features an apparatus for detecting vulnerable plaque in a blood vessel. The apparatus includes an intravascular probe having proximal and distal ends. A slip ring having a stationary portion and a spinning portion is at the proximal end. An ultrasound transceiver board is mechanically coupled to the spinning portion of the slip ring for communication with an ultrasound transducer, also within the probe. A transmission line extends between the ultrasound transducer and the ultrasound transceiver board.

In some embodiments, the apparatus also includes a pair of optical fibers extending distally from the proximal end of the probe; and an optical bench for receiving the optical fibers.

In other embodiments, the transceiver board includes an RF circuit for providing RF energy to the ultrasound transducer, and for receiving RF energy and extracting information therefrom.

Other embodiments includes those in which a power supply is coupled to the stationary portion of the slip ring for providing power to the RF circuit on the ultrasound transceiver board, and those in which a processor is coupled to the stationary portion of the slip ring for receiving data from the ultrasound transceiver board.

In another aspect, the invention features a method for detecting vulnerable plaque. The method includes inserting a catheter containing an ultrasound transducer into a blood vessel; spinning the ultrasound transducer within the catheter; and concurrent with spinning the ultrasound transducer, spinning a source of RF energy for the ultrasonic transducer.

In some practices, the method also includes coupling power from a power source to the source of RF energy, with the power source being one that can rotate relative to the source of RF power for the ultrasound transducer. Typically, relative rotation would include having the power source be in a stationary reference frame and having the catheter rotate, so that if one viewed the power source from the rotating reference frame of the catheter, it would appear to be rotating. Such coupling of power can include coupling power from a power source to the source of RF power coupling power across a slip ring.

In yet other practices, the method includes receiving a signal from the ultrasound transducer; extracting information from the received signal; encoding the extracted information onto a digital signal; and coupling the digital signal to a processor that rotates relative to the ultrasound transducer.

As used herein, “infrared” means infrared, near infrared, intermediate infrared, far infrared, or extreme infrared.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description, the claims, and the following figures, in which:

### DESCRIPTION OF DRAWINGS

FIG. 1A is a cross-sectional view of an intravascular probe with an guidewire lumen in a distal end of a catheter;

FIG. 1B is another cross-sectional view of the intravascular probe of FIG. 1A with a rotating core and a rigid coupling between an optical bench and an ultrasound transducer;

FIG. 1C is a cross-sectional view of an implementation of the intravascular probe of FIG. 1B with a single optical fiber;

FIG. 2 is a cross-sectional view of an intravascular probe with a rotating core and a flexible coupling between an optical bench and ultrasound transducer;

FIGS. 3A-B show top and side cross-sectional views of laterally adjacent unidirectional optical bench and ultrasound transducer in an intravascular probe with a rotating core;

FIG. 4 is a cross-sectional view of an intravascular probe with a rotating core and laterally adjacent opposing optical bench and ultrasound transducer;

FIG. 5 is a cross-sectional view of an intravascular probe with a fixed core, an optical bench with a radial array of optical fibers, and a radial array of ultrasound transducers;

FIGS. 6A-B compare transverse cross-sectional views of catheters with rotating and fixed cores;

FIG. 7 shows an ultrasound transceiver board at the proximal end of the catheter; and

FIG. 8 shows details of the ultrasound transceiver board

### DETAILED DESCRIPTION

The vulnerability of a plaque to rupture can be assessed by detecting a combination of attributes such as macrophage presence, local temperature rise, and a lipid-rich pool covered by a thin fibrous cap. Some detection modalities are only suited to detecting one of these attributes.

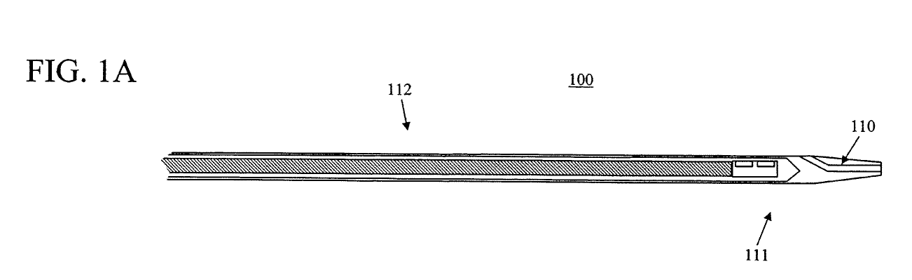

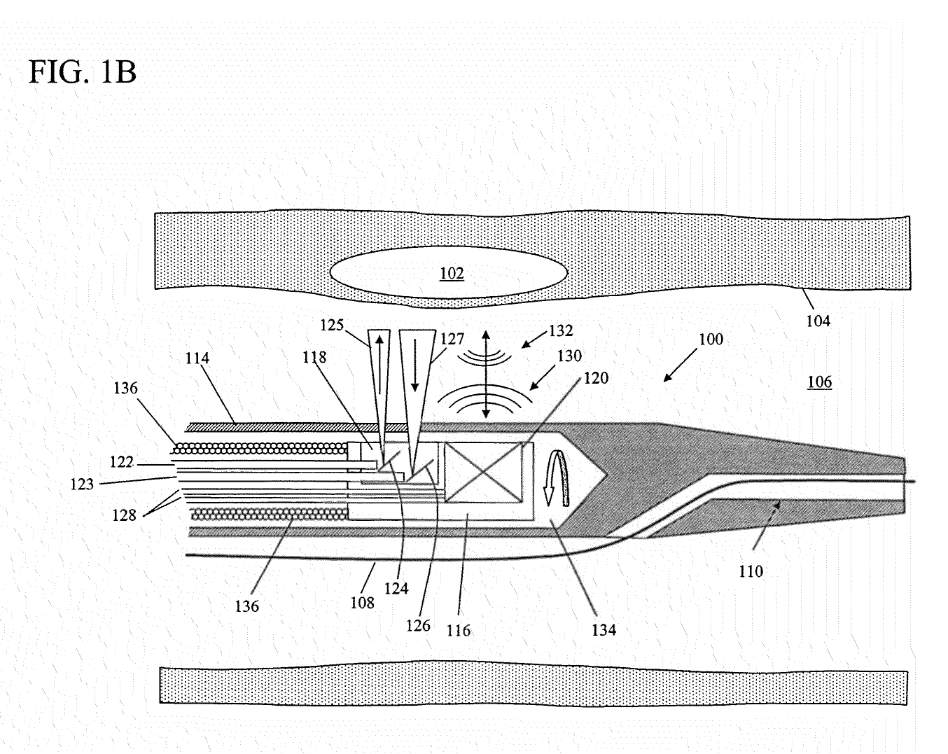

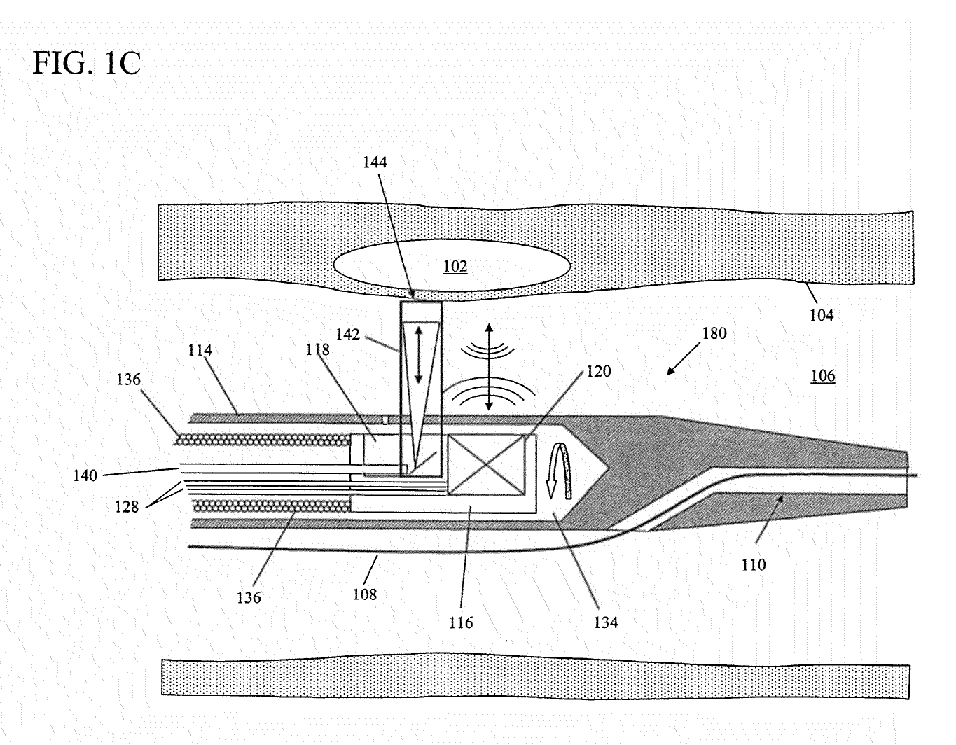

FIGS. 1A-1B show an embodiment of an intravascular probe 100 that combines two detection modalities for identifying vulnerable plaque 102 in an arterial wall 104 of a patient. The combination of both chemical analysis, using infrared spectroscopy to detect lipid content, and morphometric analysis, using IVUS to detect cap thickness, enables greater selectivity in identifying potentially vulnerable plaques than either detection modality alone. These two detection modalities can achieve high sensitivity even in an environment containing whole blood.

Referring to FIGS. 1A and 1B, an intravascular probe 100 includes a catheter 112 with a guidewire lumen 110 at a distal end 111 of the catheter 112. An outer layer of the catheter 112 features a sheath 114, best seen in FIG. 1B, composed of a material that transmits infrared light, for example a polymer. The intravascular probe 100 can be inserted into a lumen 106 of an artery using a guidewire 108 that is threaded through the guidewire lumen 110.

A delivery fiber 122 and a collection fiber 123 extend between proximal and distal ends of the catheter 112. An optical bench 118 holds the distal ends of both the collection fiber 123 and the delivery fiber 122. A housing 116 is located at the distal end of the catheter 112 houses both the optical bench 118 and one or more ultrasound transducers 120.

A light source (not shown) couples light into a proximal end of the delivery fiber 122. The delivery fiber guides this light to a delivery mirror 124 on the optical bench 118, which redirects the light 125 towards the arterial wall 104. A collection mirror 126, also on the optical bench 118, redirects light 127 scattered from various depths of the arterial wall 104 into the distal end of the collection fiber 123. Other beam redirectors can be used in place of delivery mirror 124 and collection mirror 126 (e.g., a prism or a bend in the optical fiber tip).

A proximal end of collection fiber 123 is in optical communication with an optical detector (not shown). The optical detector produces an electrical signal that contains a spectral signature indicating the composition of the arterial wall 104, and in particular, whether the composition is consistent with the presence of lipids found in a vulnerable plaque 102. The spectral signature in the electrical signal can be analyzed using a spectrum analyzer (not shown) implemented in hardware, software, or a combination thereof.

Alternatively, in an implementation shown in FIG. 1C, an intravascular probe 180 uses a single optical fiber 140 in place of the delivery fiber 122 and the collection fiber 123. By collecting scattered light directly from the intraluminal wall 104, one avoids scattering that results from propagation of light through blood within the lumen 106. As a result, it is no longer necessary to provide separate collection and delivery fibers. Instead, a single fiber 140 can be used for both collection and delivery of light using an atraumatic light-coupler 142. Referring to FIG. 1C, the atraumatic light-coupler 142 rests on a contact area 144 on the arterial wall 104. When disposed as shown in FIG. 1C, the atraumatic light-coupler 142 directs light traveling axially on the fiber 140 to the contact area 144. After leaving the atraumatic light-coupler 142, this light crosses the arterial wall 104 and illuminates structures such as any plaque 102 behind the wall 104. These structures scatter some of the light back to the contact area 144, where it re-emerges through the arterial wall 104. The atraumatic light-coupler 142 collects this re-emergent light and directs it into the fiber 140. The proximal end of the optical fiber 144 can be coupled to both a light source and an optical detector (e.g., using an optical circulator).

The ultrasound transducer 120, which is longitudinally adjacent to the optical bench 118, directs ultrasound energy 130 towards the arterial wall 104, and receives ultrasound energy 132 reflected from the arterial wall 104. Using time multiplexing, the ultrasound transducer 120 can couple both the transmitted 130 and received 132 ultrasound energy to an electrical signal carried on a transmission line 128. For example, during a first time interval, an electrical signal carried on the transmission line 128 causes the ultrasound transducer 120 to emit a corresponding ultrasound signal. Then during a second time interval, after the ultrasound signal has reflected from the arterial wall, the ultrasound transducer 120 produces an electrical signal carried on the transmission line 128. This electrical signal corresponds to the received ultrasound signal. The received electrical signal can be used to reconstruct the shape of the arterial wall, including cap thickness of any plaque 102 detected therein.

In some embodiments, multiple ultrasound transducers 120 are mounted adjacent to the optical bench 118. These multiple transducers are oriented to concurrently illuminate different circumferential angles. An advantage of such a configuration is that one can obtain the same resolution at a lower spin rate as a single transducer embodiment could achieve at a higher spin rate.

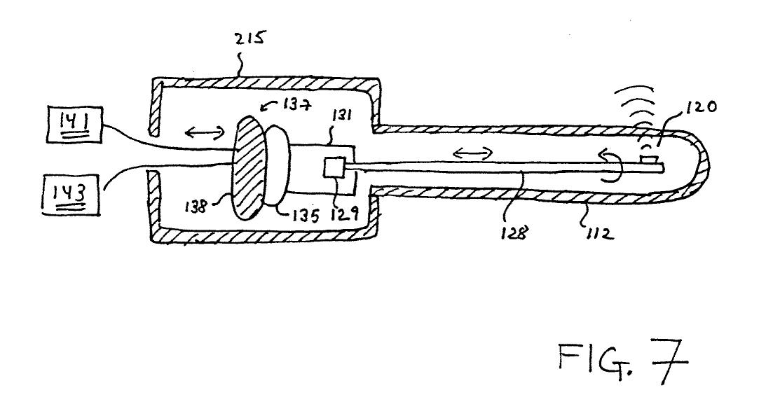

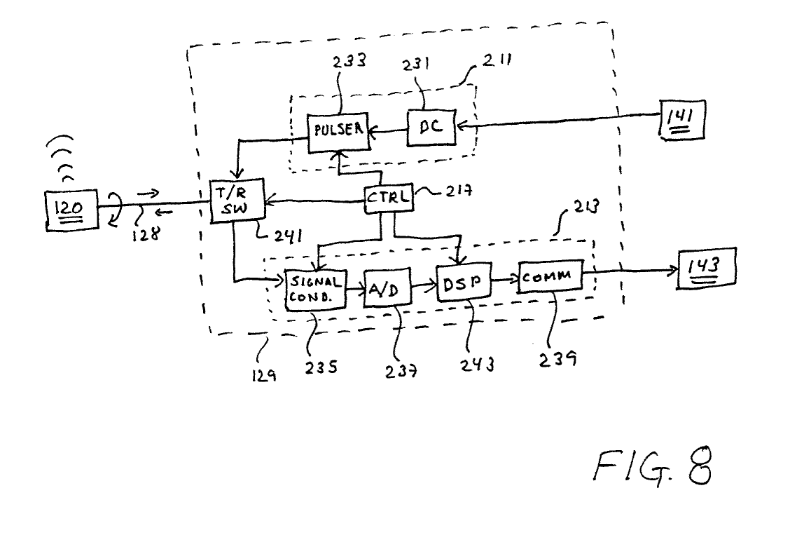

The signals carried on the transmission line 128 propagate between the transducer 120 and an RF circuit 129 mounted on an ultrasound transceiver board 131 at the proximal end of the catheter 112, as shown in FIG. 7. Referring to FIG. 8, the RF circuit 129 includes a transmitting portion 211 for generating an RF signal for transmission to the transducer 120, and a receiving portion 213 for receiving a second RF signal from the transducer 120, extracting information from that second RF signal, converting that extracted information into digital form suitable for further processing by a processor 143 outside the probe 100. The RF circuit 129 also includes control logic 217 for controlling the operation of the transmitting and receiving portions 211, 213 and for providing that information to the processor 143 either by transmitting digital signals across the slip ring 137 or by a wireless link. The transceiver board 131 is coupled to a spinning portion 135 of a slip ring 137. As a result, the entire transceiver board 131, including all components mounted thereon, is free to spin.

Referring back to FIG. 7, a pull-back-and-rotate unit 215 engages the proximal end of the catheter 112 and a stationary portion 138 of the slip ring 137. As a result, the stationary portion 138 of the slip ring 137 can translate along the axis of the catheter 112 but cannot spin. However, the spinning portion 135 of the slip ring 137, the transceiver board 131 and all components mounted thereon, the transducer 120, and the transmission line 128, are all free to both spin about and translate along the axis of the catheter 112. A suitable pull-back-and-rotate unit 215 is described in co-pending U.S. application Ser. No. 11/875,603, filed on Oct. 19, 2007, the contents of which are herein incorporated by reference.

Referring back to FIG. 8, the transmitting portion of 211 of the RF circuit 129 includes a DC converter 231 for stepping up a DC voltage provided by the power source 141. Low voltage outputs of the converter 231 provide power for other components of the circuit 129. A high voltage output is made available to a pulser 233. In response to controls signals provided by the control portion 239, the pulser 233 generates bipolar high-voltage pulses to drive the transducer 120. These pulses are placed on the transmission line 128 by a transmit/receive switch 241 controlled by the control logic 217. Typical pursers 233 include half-H bridges made using DMOS technology that are driven by low voltage pulses provided by the control logic 217.

Following transmission of a pulse, the control logic 217 switches the T/R switch 241 from transmit mode into receive mode, thereby making an echo signal available to the receiving portion 213.

The receiving portion 213 includes a signal conditioning unit 235 for receiving an RF signal from the transmission line 128 and transforming that signal into a form suitable for processing by an A/D converter 237 in electrical communication with the signal conditioning unit 235. Typical operations carried out by the signal conditioning unit 235 include amplification and filtering operations. The parameters associated with operations carried out by the signal conditioning unit 235 are provided by control signals from the control logic 217. Such control signals include signals specifying gain, compensation, and clock pulses.

The receiving portion 213 also includes a communication interface 239 for receiving digital signals from the A/D converter 237 and providing those signals to the processor 143. The receiving portion 213 also includes a digital signal processor 243 for further processing the signal received from the A/D converter 237. The additional signal processing steps can include additional filtering, decimation, ring-down suppression, and envelope detection. The resulting decimated data, which can be as much as two orders of magnitude less than the original data, is then provided to a communication interface 239 for transmission to the external processor using conventional communication protocols.

The stationary portion 138 of the slip ring 137 is coupled to a power supply 141 that provides power to the spinning RF circuit 129. The configuration shown in FIG. 7 thus avoids having RF energy crossing from the stationary portion 138 to the spinning portion 135 of the slip ring 137. This configuration thus reduces noise and electromagnetic interference associated with having RF energy crossing the slip ring 137. In addition, the configuration shown in FIG. 7, in which the transceiver board 131 is disposed distal to the slip ring 137, simplifies the design of the slip ring 137, and in fact permits the use of “off-the-shelf” slip rings.

Inside the sheath 114 is a transmission medium 134, such as saline or other fluid, surrounding the ultrasound transducer 120 for improved acoustic transmission. The transmission medium 134 is also transparent to the infrared light emitted from the optical bench 118.

A torque cable 126 attached to the housing 116 surrounds the optical fibers 122 and the wires 128. A motor (not shown) rotates the torque cable 126, thereby causing the housing 116 to rotate. This feature enables the intravascular probe 100 to circumferentially scan the arterial wall 104 with light 124 and ultrasound energy 130.

During operation, the intravascular probe 100 is inserted along a blood vessel, typically an artery, using the guidewire 108. In one practice the intravascular probe 100 is inserted in discrete steps with a complete rotation occurring at each such step. In this case, the optical and ultrasound data can be collected along discrete circular paths. Alternatively, the intravascular probe 100 is inserted continuously, with axial translation and rotation occurring simultaneously. In this case, the optical and ultrasound data are collected along continuous helical paths. In either case, the collected optical data can be used to generate a three-dimensional spectral map of the arterial wall 104, and the collected ultrasound data can be used to generate a three-dimensional morphological map of the arterial wall 104. A correspondence is then made between the optical and ultrasound data based on the relative positions of the optical bench 118 and the ultrasound transducer 120. The collected data can be used in real-time to diagnose vulnerable plaques, or identify other lesion types which have properties that can be identified by these two detection modalities, as the intravascular probe 100 traverses an artery. The intravascular probe 100 can optionally include structures for carrying out other diagnostic or treatment modalities in addition to the infrared spectroscopy and IVUS diagnostic modalities.

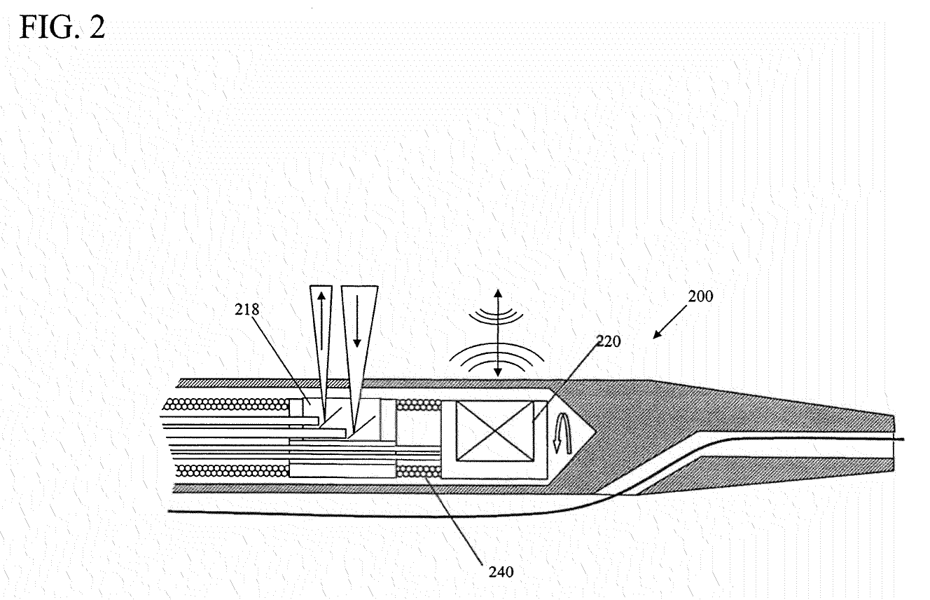

FIG. 2 is a cross-sectional view of a second embodiment of an intravascular probe 200 in which a flexible coupling 240 links an optical bench 218 and an ultrasound transducer 220. When a catheter is inserted along a blood vessel, it may be beneficial to keep any rigid components as short as possible to increase the ability of the catheter to conform to the shape of the blood vessel. Intravascular probe 200 has the advantage of being able to flex between the optical bench 218 and the ultrasound transducer 220, thereby enabling the intravascular probe 200 to negotiate a tortuous path through the vasculature. However, the optical and ultrasound data collected from intravascular probe 200 may not correspond as closely to one another as do the optical and ultrasound data collected from the intravascular probe 100. One reason for this is that the optical bench 218 and the ultrasound transducer 220 are further apart than they are in the first embodiment of the intravascular probe 100. Therefore, they collect data along different helical paths. If the catheter insertion rate is known, one may account for this path difference when determining a correspondence between the optical and ultrasound data; however, the flexible coupling 240 between the optical bench 218 and the ultrasound transducer 220 may make this more difficult than it would be in the case of the embodiment in FIG. 1A.

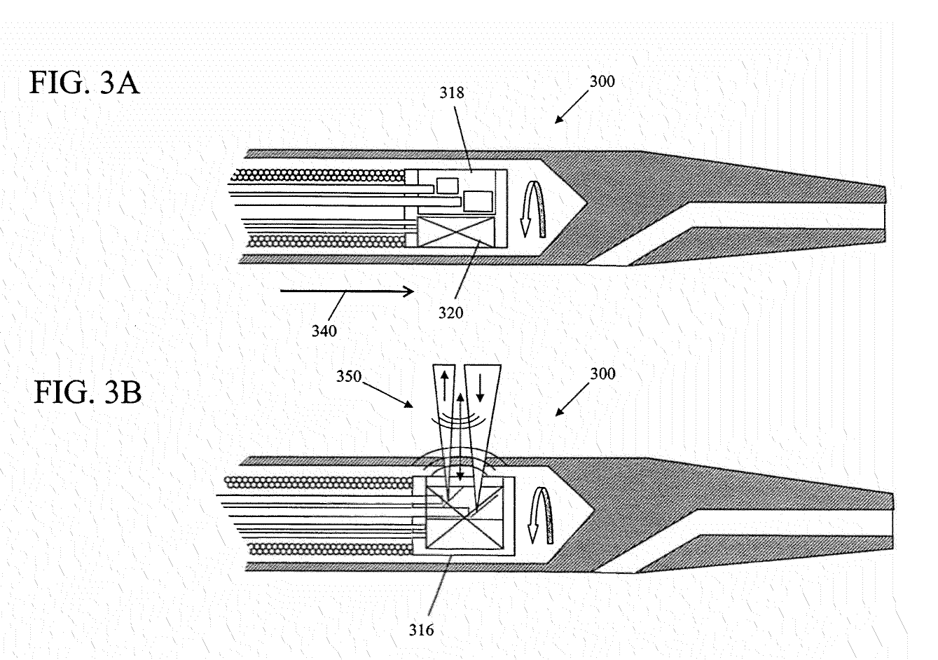

FIGS. 3A and 3B show cross-sectional views of a third embodiment in which the intravascular probe 300 has an optical bench 318 and an ultrasound transducer 320 that are laterally adjacent such that they emit light and ultrasound energy, respectively, from the same axial location with respect to a longitudinal axis 340 of the sheath 314. FIG. 3A shows the top view of the emitting ends of the optical bench 318 and ultrasound transducer 320. FIG. 3B is a side view showing the light and ultrasound energy emitted from the same axial location, so that as the housing 316 is simultaneously rotated and translated, the light and ultrasound energy 350 trace out substantially the same helical path. This facilitates matching collected optical and ultrasound data. A time offset between the optical and ultrasound data can be determined from the known rotation rate.

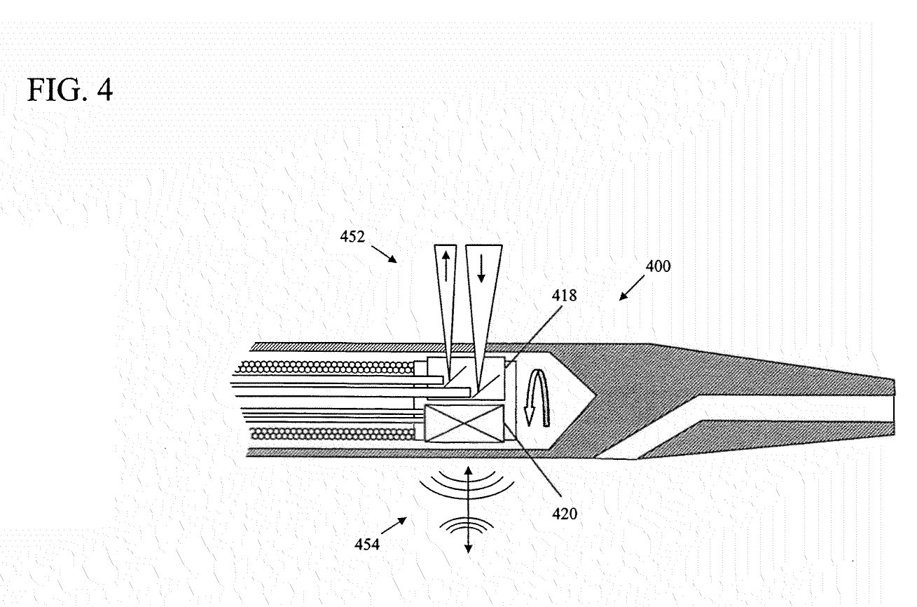

FIG. 4 is a cross-sectional view of a fourth embodiment in which intravascular probe 400 has a laterally adjacent and opposing optical bench 418 and ultrasound transducer 420 as described in connection with FIGS. 3A and 3B. However, in this embodiment, light 452 is emitted on one side and ultrasound energy 454 is emitted on an opposite side. This arrangement may allow intravascular probe 400 to have a smaller diameter than intravascular probe 300, depending on the geometries of the optical bench 418 and ultrasound transducer 420. A smaller diameter could allow an intravascular probe to traverse smaller blood vessels.

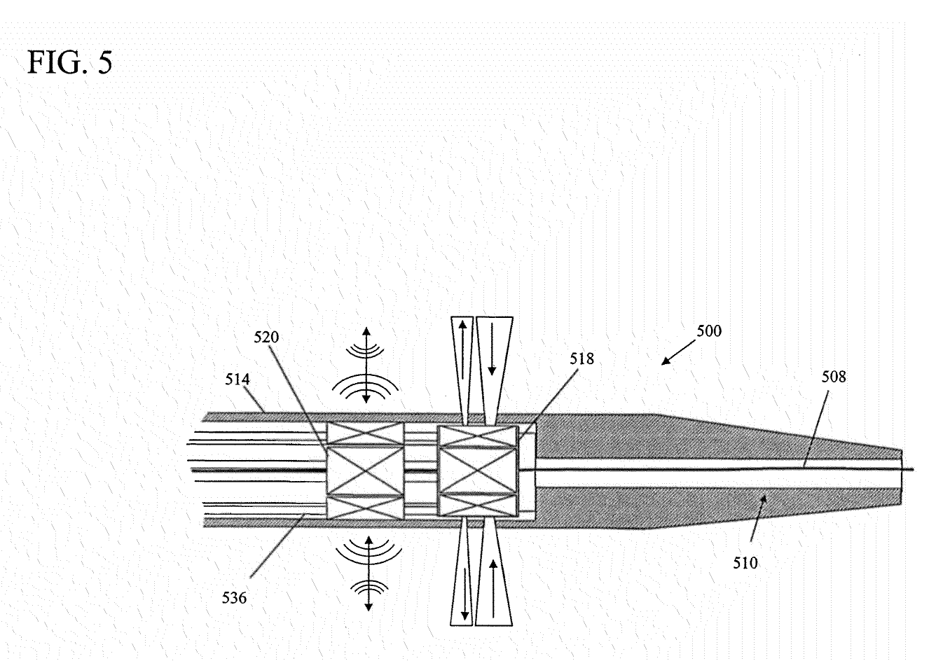

FIG. 5 is a cross-sectional view of a fifth embodiment in which intravascular probe 500 has a fixed core 536, a radial array of optical couplers 518, and a radial array of ultrasound transducers 520. The fifth embodiment, with its fixed core 536, is potentially more reliable than previous embodiments, with their rotating cores. This is because the fifth embodiment lacks moving parts such as a torque cable. Lack of moving parts also makes intravascular probe 500 safer because, should the sheath 514 rupture, the arterial wall will not contact moving parts.

The intravascular probe 500 can collect data simultaneously in all radial directions thereby enhancing speed of diagnosis. Or, the intravascular probe 500 can collect data from different locations at different times, to reduce potential crosstalk due to light being collected by neighboring optical fibers or ultrasound energy being collected by neighboring transducers. The radial resolution of spectral and/or morphological maps will be lower than the maps created in the embodiments with rotating cores, although the extent of this difference in resolution will depend on the number of optical fibers and ultrasound transducers. A large number of optical fibers and/or ultrasound transducers, while increasing the radial resolution, could also make the intravascular probe 500 too large to fit in some blood vessels.

Intravascular probe 500 can be inserted through a blood vessel along a guidewire 508 that passes through a concentric guidewire lumen 510. Inserting a catheter using a concentric guidewire lumen 510 has advantages over using an off-axis distal guidewire lumen 110. One advantage is that the guidewire 508 has a smaller chance of becoming tangled. Another advantage is that, since a user supplies a load that is coaxial to the wire during insertion, the concentric guidewire lumen 510 provides better trackability. The concentric guidewire lumen 510 also removes the guidewire 508 from the field of view of the optical fibers and ultrasound transducers.

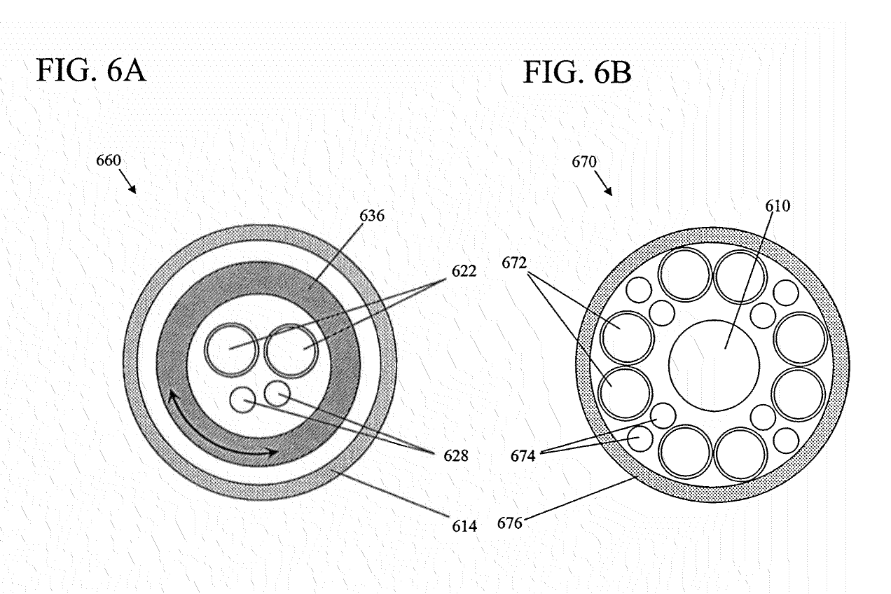

The intravascular probes include a catheter having a diameter small enough to allow insertion of the probe into small blood vessels. FIGS. 6A and 6B compare transverse cross-sectional views of catheters from embodiments with rotating cores (FIGS. 1-4) and fixed cores (FIG. 5).

The rotating core catheter 660, shown in FIG. 6A, includes a single pair of optical fibers 622, for carrying optical signals for infrared spectroscopy, and a single pair of wires 628, for carrying electrical signals for IVUS, within a hollow torque cable 636. The diameter of the sheath 614 of catheter 660 is limited by the size of the torque cable 636.

The fixed core catheter 670, shown in FIG. 6B, has four optical fiber pairs 672, and four wire pairs 674, for carrying optical signals and electrical IVUS signals, respectively, from four quadrants of the arterial wall. While no torque cable is necessary, the sheath 676 of catheter 670 should have a diameter large enough to accommodate a pair of optical fibers 672 and a pair of wires 674 for each of the four quadrants, as well as a concentric guidewire lumen 610.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. An apparatus for detecting vulnerable plaque in a blood vessel, the apparatus comprising:
an intravascular probe having a proximal end and a distal end;
a slip ring at the proximal end of the probe, the slip ring having a stationary portion and a spinning portion;
an ultrasound transducer mounted within the intravascular probe;
an ultrasound transceiver board mechanically coupled to the spinning portion of the slip ring; and
a transmission line extended between the ultrasound transducer and the ultrasound transceiver board.

2. The apparatus of claim 1, further comprising:
a pair of optical fibers extending distally from the proximal end of the probe; and
an optical bench for receiving the optical fibers.

3. The apparatus of claim 2, wherein the transceiver board comprises an RF circuit for providing RF energy to the ultrasound transducer, and for receiving RF energy and extracting information therefrom.

4. The apparatus of claim 1, further comprising a power supply coupled to the stationary portion of the slip ring for providing power to the RF circuit on the ultrasound transceiver board.

5. The apparatus of claim 1, further comprising a processor coupled to the stationary portion of the slip ring for receiving data from the ultrasound transceiver board.

6. A method for detecting vulnerable plaque, the method comprising:
inserting a catheter containing an ultrasound transducer into a blood vessel;
spinning the ultrasound transducer within the catheter; and
concurrent with spinning the ultrasound transducer, spinning a source of RF energy for the ultrasonic transducer.

7. The method of claim 6, further comprising coupling power from a power source to the source of RF energy, wherein the power source rotates relative to the source of RF power for the ultrasound transducer.

8. The method of claim 7, wherein coupling power from a power source to the source of RF power comprises coupling power across a slip ring.

9. The method of claim 6, further comprising:
receiving a signal from the ultrasound transducer;
extracting information from the received signal;
encoding the extracted information onto a digital signal; and
coupling the digital signal to a processor that rotates relative to the ultrasound transducer.
